(19) United States
(12) Patent Application Publication
(10) Pub. No.: US 2025/0332063 A1
(43) Pub. Date: Oct. 30, 2025

(54) ABDOMINAL THRUST DEVICE

(71) Applicant: Kuo Huang YANG, Taipei (TW)

(72) Inventor: Kuo Huang YANG, Taipei (TW)

(21) Appl. No.: 19/250,244

(22) Filed: Jun. 26, 2025

Related U.S. Application Data
(63) Continuation of application No. PCT/CN2025/090615, filed on Apr. 23, 2025.

(30) Foreign Application Priority Data
Apr. 26, 2024 (CN) 202410510158.7

(51) Int. Cl. A61H 31/00

(52) U.S. Cl. CPC A61H 31/00; A61H 2201/0149; A61H 2201/1253; A61H 2201/1619; A61H 2201/168

(57) ABSTRACT

The present disclosure discloses an abdominal thrust device fixed to an upper edge of a chair back, comprising a fastener, at least one fastening element, and a pressing member. The fastener further comprises a pressure-bearing portion, and the fastener is fixed to the chair back structure by the at least one fastening element so as to have the pressure-bearing portion tightly bear against the chair back structure. The pressing member has a rounded blunt shape and is disposed at a top portion of the fastener.

### FIELD OF TECHNOLOGY

The present disclosure relates to an abdominal thrust device, and more particularly to an abdominal thrust device for self-rescue in cases of choking.

### BACKGROUND

Abdominal thrusts, also known as the Heimlich maneuver, are a first aid technique used to remove obstructions from the upper airway. In cases where a person is choking while alone, though it is possible to expel a foreign object lodged in the trachea by pressing the upper abdomen against the chair back to generate an impact force, similarly to the Heimlich maneuver; however, due to the fact that the upper edge structures of most chair backs are narrow and slender, the applied force to the abdomen tends to disperse. As a result, the pressure generated by the abdominal thrusts often cannot be focused effectively, thereby delaying emergency rescue.

### SUMMARY

The present disclosure provides an abdominal thrust device to address the deficiencies of the prior art.

In view of the foregoing issues, the present disclosure provides an abdominal thrust device that can be conveniently fixed to an upper edge of a chair back and is not easily dislodged, while also enabling focusing the abdominal thrust pressure.

To achieve the above objective, the present disclosure provides an abdominal thrust device comprising the following aspects:

An abdominal thrust device, fixed to a chair back structure, characterized in that comprising:
a fastener, having a pressure-bearing portion, the fastener being fixed to the chair back structure by at least one fastening element so as to have the pressure-bearing portion tightly bear against the chair back structure; and
a pressing member, having an upwardly protruding rounded blunt shape, disposed at a top portion of the fastener.

According to an embodiment of the present disclosure, wherein the at least one fastening element is at least one selected from a group consisting of a screw, an adhesive, a stopper portion, a spring, an extending clamping portion, and a protruding portion.

According to an embodiment of the present disclosure, wherein the fastener comprises a fixing plate, the pressing member is disposed at a top portion of the fixing plate, and a bottom of the fixing plate serves as the pressure-bearing portion.

According to an embodiment of the present disclosure, wherein the pressing member has a shape of a human fist.

According to an embodiment of the present disclosure, wherein a maximum width of a horizontal cross-section of the pressing member is, for example, greater than or equal to 8 cm, preferably greater than or equal to 10 cm, and more preferably greater than or equal to 12 cm, and the horizontal cross-section is parallel to an upper edge where the pressure-bearing portion tightly bears the chair back structure.

According to an embodiment of the present disclosure, the abdominal thrust device further comprises a soft material that covers at least a portion of the pressing member.

According to an embodiment of the present disclosure, wherein the top portion of the fastener has a first engaging portion, and a bottom of the pressing member has a second engaging portion, the first engaging portion engaging with the second engaging portion to fix the pressing member to the top portion of the fastener.

According to an embodiment of the present disclosure, wherein the fastener, the pressing member, or the at least one fastening element are further connected to each other by a chain or rope.

According to an embodiment of the present disclosure, wherein the fastener further comprises a first side extension wall, the first side extension wall being connected to a side edge of the pressure-bearing portion and disposed perpendicular to the pressure-bearing portion;

the first side extension wall and a remaining portion of the fastener other than the first side extension wall are either integrally formed or each is a separate component connected to each other.

According to an embodiment of the present disclosure, wherein the fastener further comprises a second side extension wall, the second side extension wall being connected to another side edge of the pressure-bearing portion opposite to the side edge and disposed perpendicular to the pressure-bearing portion;

the second side extension wall and a remaining portion of the fastener other than the second side extension wall are either integrally formed or each is a separate component connected to each other.

According to an embodiment of the present disclosure, wherein the at least one fastening element is at least one selected from a group consisting of a screw, an adhesive, a stopper portion, and a spring.

According to an embodiment of the present disclosure, wherein the chair back structure is a chair back crest rail;

the fastener further comprises a horizontal extension wall, the horizontal extension wall being connected to the first side extension wall and disposed parallel to the pressure-bearing portion.

According to an embodiment of the present disclosure, wherein the at least one fastening element is an extending clamping portion or a spring, the extending clamping portion or the spring connecting the first side extension wall and the horizontal extension wall.

According to an embodiment of the present disclosure, wherein the fastener is a C-shaped clamp.

According to an embodiment of the present disclosure, wherein the at least one fastening element is a screw.

According to an embodiment of the present disclosure, wherein the C-shaped clamp elastically clamps the chair back structure.

According to an embodiment of the present disclosure, wherein the at least one fastening element is a protruding portion, and the C-shaped clamp bears against the chair back structure by the protruding portion.

According to an embodiment of the present disclosure, wherein the at least one fastening element and the fastener are integrally formed.

According to an embodiment of the present disclosure, wherein the C-shaped clamp comprises at least one side extension wall.

According to an embodiment of the present disclosure, wherein the fastening element is a screw, and an end of the screw is provided with a stopper plate, the screw bearing against the chair back structure via the stopper plate.

### BRIEF DESCRIPTION OF THE DRAWINGS

FIG. 1 shows a schematic view illustrating an abdominal thrust device according to a first embodiment of the present disclosure.

FIG. 2A and FIG. 2B show schematic views illustrating different perspectives of FIG. 1.

FIG. 3 shows a schematic view illustrating an abdominal thrust device according to a second embodiment of the present disclosure.

FIG. 4A and FIG. 4B show side schematic views illustrating an abdominal thrust device according to a third embodiment of the present disclosure.

FIG. 5 shows a side schematic view illustrating an abdominal thrust device according to a fourth embodiment of the present disclosure.

FIG. 6A, FIG. 6B, FIG. 6C and FIG. 6D show schematic views illustrating an abdominal thrust device according to a fifth embodiment of the present disclosure.

FIG. 7 shows a side schematic view illustrating an abdominal thrust device according to a sixth embodiment of the present disclosure.

FIG. 8A and FIG. 8B show schematic views illustrating an abdominal thrust device according to a seventh embodiment of the present disclosure.

### LIST OF REFERENCE NUMERALS

10: abdominal thrust device
11: fastener
12: pressure-bearing portion
13: fixing plate
14: first side extension wall
15: second side extension wall
16: horizontal extension wall
17: first clamping portion
18: second clamping portion
21: pressing member
22: screw hole
23: soft material
31: screw
311: bump
312: stopper plate
32: adhesive
33: stopper portion
34: spring
35: extending clamping portion
36: protruding portion
41: first engaging portion
42: second engaging portion
100: chair back structure
101: upper edge of the chair back structure
102: chair back crest rail
103: chair back crest rail
A1: symmetry axis
A2: symmetry axis
F1: force
F2: reaction force
W: maximum width

### DETAILED DESCRIPTION

Specific embodiments of the present disclosure are described below with reference to the drawings, in which identical or similar parts are denoted by the same reference numerals. It should be noted that the following embodiments and examples are intended to illustrate the content of the present disclosure, and are not intended to limit the scope of the present disclosure.

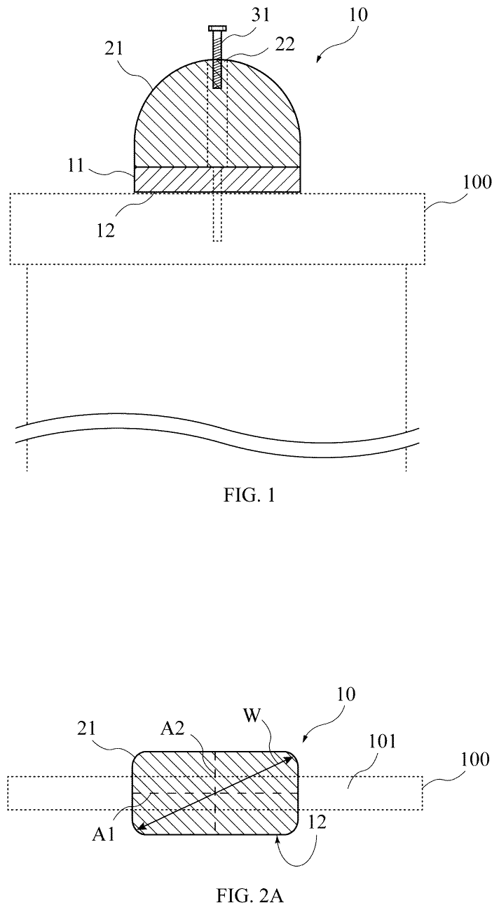

FIG. 1 shows a schematic view illustrating an abdominal thrust device according to a first embodiment of the present disclosure. In FIG. 1, an abdominal thrust device 10 is fixed to a chair back structure 100. The abdominal thrust device 10 comprises a fastener 11 and a pressing member 21.

A bottom of the fastener 11 includes a pressure-bearing portion 12. The fastener 11 is fixed to the chair back structure 100 by at least one fastening element. The fastening element passes through or engages with the fastener 11 and is inserted into or bears against the chair back structure 100 so as to have the pressure-bearing portion 12 tightly bear against the chair back structure 100. The pressing member 21 has an upwardly protruding rounded blunt shape and is disposed at a top portion of the fastener 11. In the present disclosure, the “bottom of the fastener” refers to the side of the fastener that is closer to the ground, and the “top portion of the fastener” refers to the side of the fastener opposite to the side where the pressure-bearing portion is located, that is, the side of the fastener that is away from the ground. In the present embodiment, the fastener 11 and the pressing member 21 are integrally formed. The fastening element is a screw 31, which is inserted into the chair back structure 100 through a screw hole 22 that formed through the pressing member 21 and the fastener 11, thereby fixing the fastener 11 to the chair back structure 100. However, in other embodiments, the fastening element and the fastener 11 may also be integrally formed, so as to stably fix the abdominal thrust device 10 to an upper edge of the chair back.

In the present disclosure, the method of “integral formation” may be achieved by any manner known to a person of ordinary skill in the art to which the present disclosure pertains, such as one-time injection molding, casting, stamping, line turning, or the like.

Accordingly, during use, a user's abdomen presses against the pressing member 21 of the abdominal thrust device 10 and applies a downward force so that the pressing member 21 generates an upward reaction force to impact the user's abdomen. Thus, since the abdominal thrust device 10 is stably fixed to the chair back structure 100, the user can rapidly and ergonomically repeat abdominal thrusts in a less labor-intensive manner to expel a foreign object lodged in the trachea.

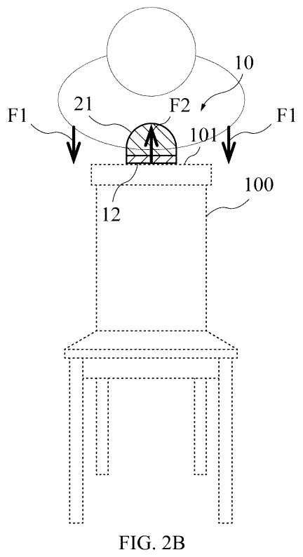

FIG. 2A and FIG. 2B show schematic views showing different perspectives of FIG. 1. FIG. 2A shows a top view of FIG. 1. The pressure-bearing portion 12 preferably has a symmetrical shape, with a symmetry axis A1 (parallel to the upper edge of the chair back structure 101) arranged horizontally. A horizontal cross-section of the pressing member 21 (which is parallel to the surface where the pressure-bearing portion 12 tightly bears the chair back structure 100, i.e., parallel to the upper edge of the chair back structure 101) has a maximum width W, which is, for example, greater than or equal to 8 cm, preferably greater than or equal to 10 cm, and more preferably greater than or equal to 12 cm. Accordingly, the pressing member 21 may provide optimal pressure for abdominal thrusts. FIG. 2B shows a schematic view showing a user using the embodiment of FIG. 1. A top portion of the pressing member 21 has an upwardly protruding arcuate shape. When a user with an upper airway obstructed with a foreign object stands behind a chair and faces the abdominal thrust device 10, the user bends over to press an upper abdominal area (a position at the bottom of the diaphragm) against the pressing member 21 and then forcibly pushes downward using the upper body. The user's body weight, along with pulling force applied by the arms (not shown, e.g., by gripping the seat of the chair), generates a downward force F1. Simultaneously, because the pressure-bearing portion 12 tightly bears against the chair back structure 100 (i.e., the upper edge of the chair back structure 101), the user's downward force F1 causes the pressing member 21 to generate an upward reaction force F2. This mechanism allows the user to repeatedly apply the reaction force F2 to the abdomen to expel a foreign object from the trachea. The upwardly protruding arcuate shape of the top portion, along with a maximum width W (see also FIG. 2A) of greater than or equal to 8 cm, of the pressing member 21 may protect the user from injury during forceful abdominal thrusts. In addition, since an excessive width of pressing member 21 may disperse the pressure applied to the abdomen, the maximum width W is preferably less than or equal to 20 cm.

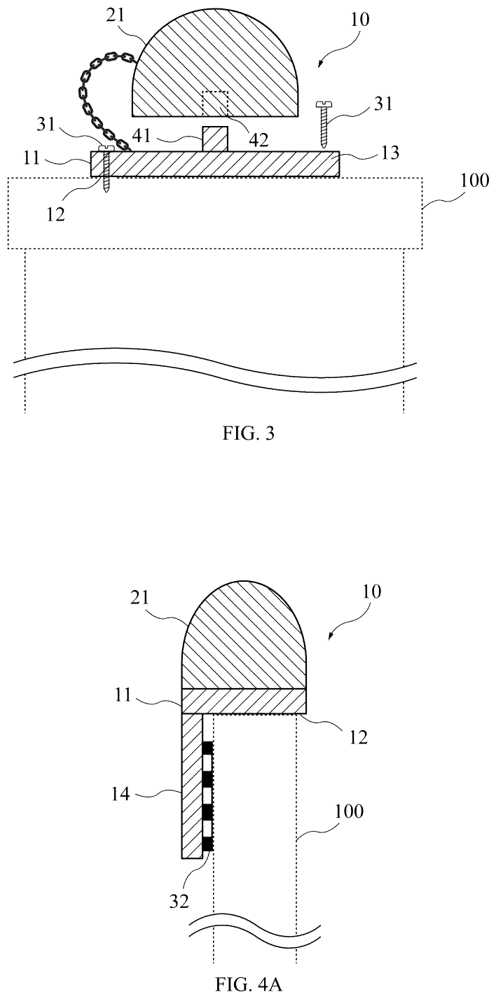

Moreover, in the present disclosure, the fastener 11 may have various configurations for installing or fixing the abdominal thrust device 10 to an upper edge of a chair back. For example, the fastener 11 and the pressing member 21 may be integrally formed or may be separate components connected to each other by mechanical or structural means. The fastener 11 may include various types of structural components, including but not limited to plate-shaped elements, arc-shaped elements, irregular-shaped elements and the like. Such a design allows for high adaptability and versatility to accommodate different chair back shapes or application needs. For example, when the fastener 11 includes a plate-shaped element (also referred to as a fixing plate in the present disclosure), a bottom of the plate-shaped element may serve as the pressure-bearing portion 12, and the width and/or area of a top portion of the plate-shaped element may be designed to be less than, equal to, or greater than the width and/or area of a bottom of the pressing member 21 (i.e., the side of the pressing member 21 joined to a top portion of the fastener 11), depending on application requirements. Accordingly, it is readily understood that the fixing plate and the pressing member 21 may be integrally formed or may be separately formed and joined together. For example, FIG. 3 shows a schematic view illustrating an abdominal thrust device according to a second embodiment of the present disclosure, wherein the fastener 11 includes a fixing plate 13. The pressing member 21 is disposed at a top portion of the fixing plate 13, and each of the fixing plate 13 and the pressing member 21 is a separate component. In this embodiment, a width and area of the top portion of the fixing plate 13 are greater than a width and area of the bottom of the pressing member 21, thereby providing excellent stability and structural support. A bottom of the fixing plate 13 serves as the pressure-bearing portion 12 of the fastener 11. The fastening element is at least one screw 31, which has a pointed tip at its end that may be drilled into the chair back structure 100, thereby fixing the fixing plate 13 to the chair back structure 100. A top portion of the fastener 11 has a first engaging portion 41, and a bottom of the pressing member 21 has a second engaging portion 42. In this embodiment, the first engaging portion 41 and the second engaging portion 42 form a snap-fit structure, in which the first engaging portion 41 engages the second engaging portion 42 to fix the pressing member 21 to the top portion of the fastener 11. With the snap-fit structure, the user may conveniently disassemble or assemble the fastener 11 and the pressing member 21 as desired, thereby providing the user with an option to remove the pressing member 21 for improved comfort to the back. In other embodiments, the first engaging portion 41 and the second engaging portion 42 may form a structure of internal screw threads and external screw threads or a barb structure, which will be described in detail later. In addition, the fastener 11 and the pressing member 21 may further be linked by a chain or rope to prevent insufficient time to assemble the two during an emergency. For example, the fastener 11 and the pressing member 21 may each be a separate component and remain unassembled under normal conditions. In an emergency, the user or others may quickly grasp the components via the chain or rope and assemble them to immediately use the abdominal thrust device 10 of the present disclosure.

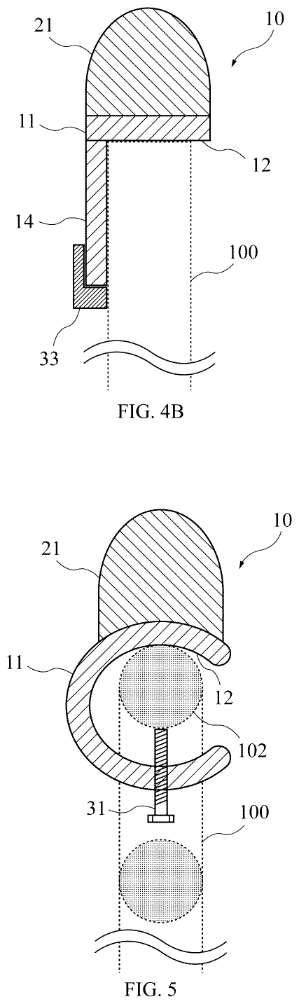

FIG. 4A and FIG. 4B show side schematic views showing the abdominal thrust device according to the third embodiment of the present disclosure. As shown in FIG. 4A, the fastener 11 includes a first side extension wall 14, which is disposed perpendicularly to the symmetry axis A2 of the pressure-bearing portion 12 (which is perpendicular to the upper edge of the chair back structure 101, please also refer to FIG. 2A) and is connected to one side edge of the pressure-bearing portion 12. Furthermore, it should be understood by a person of ordinary skill in the art that, in this embodiment, the fastener 11 may also include various types of structural components, and the first side extension wall 14 and the various types of structural components may be integrally formed, or may be two separate elements connected by means of engagement, locking, adhesion, and the like; for example, when the fastener 11 includes a plate-shaped element (e.g., a fixing plate 13), the first side extension wall 14 may be connected to one side edge of the bottom of the fixing plate 13. In the embodiment shown in FIG. 4A, the fastening element is at least an adhesive 32, which is disposed between the fastener 11 and the chair back structure 100 and fixes both via the adhesion of the adhesive 32; whereas in the embodiment shown in FIG. 4B, the fastening element is a stopper portion 33, which is connected to the chair back structure 100, and a gap is provided between the stopper portion 33 and the chair back structure 100 for the insertion of the first side extension wall 14, so that the pressure-bearing portion 12 and the first side extension wall 14 tightly bear against the chair back structure 100. As in the above-described embodiments, the stopper portion 33 and the fastener 11 may further be connected by a chain or rope, so as to allow the user to arbitrarily disassemble or assemble them.

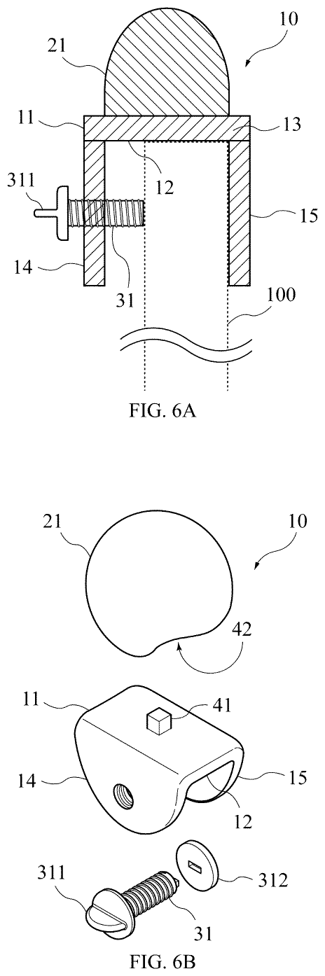

FIG. 5 shows a side schematic view showing the abdominal thrust device according to the fourth embodiment of the present disclosure. In this aspect, the fastener 11 is a “regular C-shaped” clamp and the fastening element is a screw 31. In this embodiment, the chair back structure 100 has a cylindrical chair back crest rail 102. The screw 31 preferably has a shaft with a longer length. In use, the screw 31 is rotated to penetrate the fastener 11 so that the end of the shaft bears against the chair back crest rail 102, thereby fixing the abdominal thrust device 10 without damaging the chair back structure 100.

It should be noted that, in the present disclosure, “C-shaped” refers to a broadly defined C-shaped structure, which includes but is not limited to nonlinear geometrical configurations generally in C-shape, U-shape, or others having curved, bent, or arched contours. The adjacent sides of the C-shaped structure may be optionally connected by means of smoothly curved transition, sharp angles, right angles, or obtuse angles, and may be formed as an integral curved body or a segmented structure connected via distinct fold lines, thereby forming a variably curved structure with different side lengths and geometrical connection forms. Therefore, the C-shaped clamp of the present disclosure may conform to the shape of various chair back structures and fit more securely thereagainst.

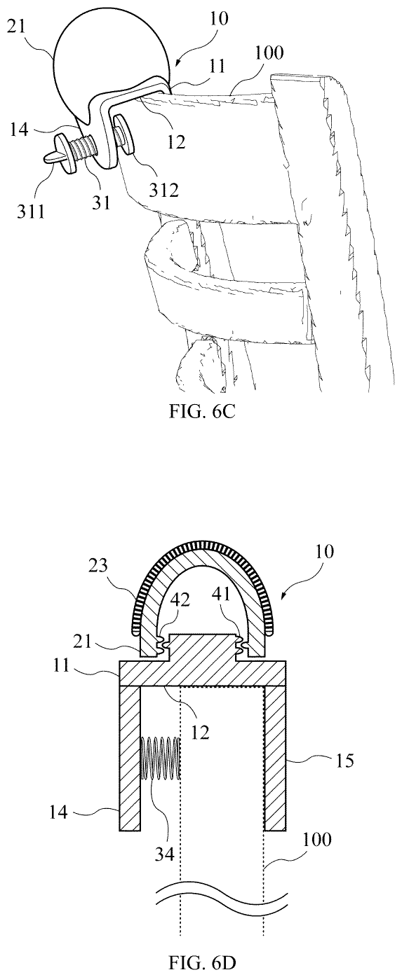

FIG. 6A, FIG. 6B, FIG. 6C and FIG. 6D show schematic diagrams illustrating a fifth embodiment of the abdominal thrust device of the present disclosure, in which the fastener 11 includes a first side extension wall 14 and a second side extension wall 15. That is, in this embodiment, the fastener 11 may be deemed as having a C-shaped structure with recessed folds and/or fold-line joints. As shown in FIG. 6A (a side view), at least one of the first side extension wall 14 and the second side extension wall 15 is vertically arranged relative to the symmetry axis A2 of the pressure-bearing portion 12 (which is perpendicular to the upper edge of the chair back structure 101; please also refer to FIG. 2A), and the first side extension wall 14 and the second side extension wall 15 are respectively connected to the two opposite side edges of the pressure-bearing portion 12. In addition, it should be understood by a person of ordinary skill in the art that in this embodiment, the fastener 11 may also include various types of structural components. Among these, at least two of the first side extension wall 14, the second side extension wall 15, and the various types of structural components may be integrally formed, and/or at least one of the elements may be a separate element connected to the others by means of engagement, locking, adhesion, and the like. For example, when the fastener 11 includes a plate-shaped element (e.g., a fixing plate 13), the first side extension wall 14 and the second side extension wall 15 may be respectively connected to the two opposite side edges of the bottom of the fixing plate 13. The first side extension wall 14, the second side extension wall 15, and the fixing plate 13 may be integrally formed; alternatively, the first side extension wall 14 and the fixing plate 13 may be integrally formed while the second side extension wall 15 is connected to the fixing plate 13 by engagement; or, the connections via engagement, or via other possible means may be exist both between the first side extension wall 14 and the fixing plate 13, and between the second side extension wall 15 and the fixing plate 13. In this embodiment, the fastening element is a screw 31, with its shaft preferably having a larger diameter. Since the distance between the first side extension wall 14 and the second side extension wall 15 in this embodiment is fixed and cannot clamp a chair back structure 100 that is thinner than that distance, rotating the screw 31 to penetrate the fastener 11 to bear against the chair back structure 100 enables securely clamping of chair backs with varying thicknesses. It should also be understood by a person of ordinary skill in the art that the fastening element in this embodiment may also include other forms such as adhesives, and is not limited to a single screw, but may vary depending on actual needs. FIG. 6B shows an exploded view illustrating the components of an embodiment of the abdominal thrust device 10, in which the pressing member 21 is an ellipsoid, and the first engaging portion 41 is used to engage with the second engaging portion 42 (not shown in the figure) at the bottom of the pressing member 21. A circular hole is predrilled at the center of the first side extension wall 14, and internal screw threads are formed on the inner wall of the circular hole. A bump 311 is provided on the outer side of the head of the screw 31, and a stopper plate 312 may be disposed at the distal end of the screw 31. Steps for use are as shown in FIG. 6C. After assembling the fastener 11 and the pressing member 21 in FIG. 6B, the user pinches the bump 311 with their fingers to rotate the screw 31 and screw it into the circular hole on the first side extension wall 14. Then, the stopper plate 312 is assembled, and the screw 31 is further rotated until the stopper plate 312 bears against the chair back structure 100. Because the longitudinal (axial) stress is distributed across the contact surface of the threads (i.e., the threads of the screw 31 and the internal screw threads of the circular hole), a secure connection is achieved between the fastener 11 and the chair back structure 100. The stopper plate 312 provides a larger contact area, thereby increasing the friction between the screw 31 and the chair back structure 100, preventing the device from shifting during abdominal thrusts. This type of clamp having a C-like shape (fastener 11) is not only adaptable to chair backs of various thicknesses, but is also highly stable even when clamped onto ergonomically designed curved chair backs as in this embodiment. As shown in FIG. 6D, in an example, the fastening element may be a spring 34. The elasticity of the spring 34 enables it to respectively bear against the first side extension wall 14 (or the second side extension wall 15) and the chair back structure 100, thereby achieving a securing effect. In this example, the first engaging portion 41 and the second engaging portion 42 may be provided as external and internal screw threads, respectively. As shown in FIG. 6D, the pressing member 21 is specifically illustrated in a cross-sectional view (a cross-section parallel to axis A2 in FIG. 2A) to show that the second engaging portion 42 at its bottom is an internal screw thread, and the first engaging portion 41 at the top portion of the fastener 11 is an external screw thread. This threaded design allows the user to conveniently rotate and detach or assemble the pressing member 21 and the fastener 11. However, apart from the aforementioned configurations, it should be understood by a person of ordinary skill in the art to which the present disclosure pertains that, in this embodiment, the first engaging portion 41 and the second engaging portion 42 may alternatively be provided as a snap-fit structure or barb structure. Additionally, at least a part of the surface of the pressing member 21 may be further covered with a layer of soft material 23. The soft material 23 may include, but is not limited to, leather, cotton, fabric, foam, silicone, rubber, or a combination thereof, to provide a more comfortable experience for the user.

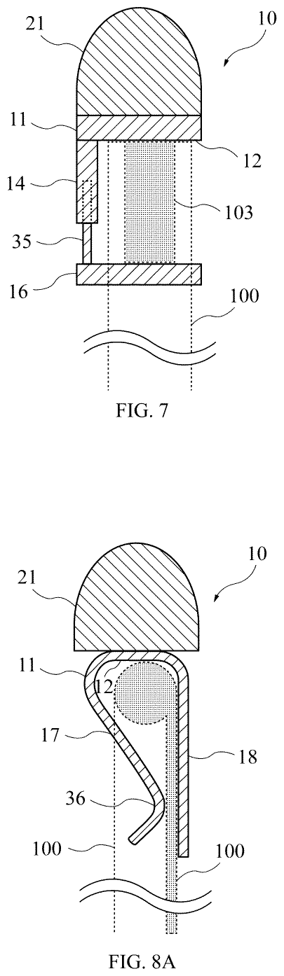

FIG. 7 shows a side schematic view illustrating a sixth embodiment of the abdominal thrust device of the present disclosure, in which the fastener 11 includes a first side extension wall 14 and a horizontal extension wall 16. The first side extension wall 14 is connected to one side edge of the pressure-bearing portion 12 and is vertically arranged relative to the pressure-bearing portion 12, while the horizontal extension wall 16 is connected to the first side extension wall 14 and is arranged parallel to the pressure-bearing portion 12. That is, in this embodiment, the fastener 11 may be deemed as having a C-shaped structure with recessed folds and/or fold-line joints. In addition, it should be understood by a person of ordinary skill in the art that in this embodiment, the fastener 11 may also include various types of structural components, and at least two of the first side extension wall 14, the horizontal extension wall 16, and the various types of structural components of the fastener 11 may be integrally formed, and/or at least one of these elements may be a separate element connected to the others by means of engagement, locking, adhesion, and the like. For example, when the fastener 11 includes a plate-shaped element (e.g., a fixing plate 13, with reference numeral of the element omitted from the figure for simplicity), the first side extension wall 14 may be connected to one side edge of the bottom of the fixing plate 13. In this embodiment, the chair back structure 100 includes a flat chair back crest rail 103. A person of ordinary skill in the art to which the present disclosure pertains should understand that the fastening element of this embodiment may be a screw 31 (not shown), which may bear against the lower edge of the chair back crest rail 103 after penetrating the horizontal extension wall 16 (please also refer to FIG. 5); alternatively, the first side extension wall 14 of this embodiment may include an extending clamping portion 35, which serves as a fastening element and connects the horizontal extension wall 16 to the lower edge of the extending clamping portion 35. By the extending clamping portion 35 parallel to the first side extension wall 14, the span of the clamp having a C-like shape (fastener 11) may be adjusted to allow the horizontal extension wall 16 to tightly bear against the lower edge of the chair back crest rail 103. The extending clamping portion 35 may further utilize the elasticity of a spring 34 or the stretch and contraction force of other materials to generate clamping force to fix the fastener 11 to the chair back crest rail 103.

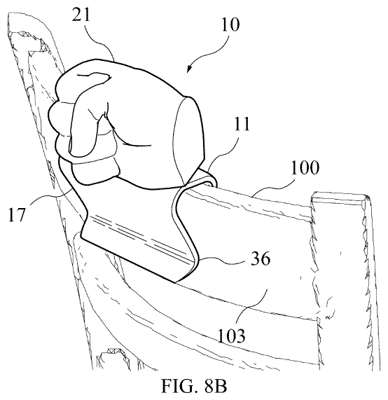

The fastener 11 may also be an elastic C-shaped clamp configured to elastically clamp the chair back structure. As described above, the C-shaped clamp, for example, may include, but is not limited to, configurations having rounded corners, sharp angles, right angles, or obtuse angles, and may be integrally formed as curved or joined via distinct fold lines. For example, FIG. 8A and FIG. 8B show schematic diagrams illustrating a seventh embodiment of the abdominal thrust device of the present disclosure, in which the fastener 11 is a C-shaped clamp with an arcuate curve, clamping the chair back structure 100 via the elasticity of its material. As shown in FIG. 8A (side view), the fastener 11 further includes a first clamping portion 17, a second clamping portion 18, and a plate-shaped element (e.g., a fixing plate 13, with reference numeral of the element omitted from the figure for simplicity). The second clamping portion 18 includes a side extension wall, and at least one of the first clamping portion 17 and the second clamping portion 18 includes at least one fastening element. In this embodiment, the fastening element is a protruding portion 36 configured to increase friction, such that the fastener 11 combined with the protruding portion 36 forms an elastic clamp, enabling convenient clamping onto chair backs of various thicknesses. In this embodiment, the fastening element and the fastener 11 may be integrally formed. As shown in FIG. 8B, the C-shaped fastener 11 made of elastic material clamps the chair back crest rail 103 of the chair back structure 100 via the protruding portion 36. The pressing member 21 may have the shape of a human fist with the hollow between the thumb and index finger of the clenched fist facing upward, disposed at the top portion of the fastener 11, thereby mimicking the effect of delivering abdominal thrusts using a fist, as in the Heimlich maneuver. In cases where a person is choking while alone or in the presence of others who are unfamiliar with the Heimlich maneuver, they may not immediately realize that the abdominal thrust device 10 of the present disclosure can be used for first aid in an emergency even if they see it. If they have to rely on user instructions to understand how to operate it, the critical first-aid time of four minutes may be wasted, thus delaying first aid-especially in the event of a language barrier that makes reading difficult. With the increasing promotion of abdominal thrust techniques in recent years, the presence of a pressing member 21 shaped like a human fist on the chair back structure 100 can, in a critical moment, help the choking person instantly recall the Heimlich maneuver and further perform self-rescue. In addition to the configurations shown in FIG. 8A and FIG. 8B, the elastic C-shaped clamp may also be formed with fold-line joints, for example, including at least one side extension wall for elastically clamping the chair back structure; and/or may include various types of structural components, including but not limited to plate-shaped elements, arc-shaped elements, irregular-shaped elements, and the like.

The foregoing is merely illustrative and not restrictive. Any equivalent modifications or alterations made without departing from the spirit and scope of the present disclosure shall be encompassed within the scope of the appended claims.

What is claimed is:

1. An abdominal thrust device, fixed to a chair back structure, characterized in that comprising:
a fastener, having a pressure-bearing portion, the fastener being fixed to the chair back structure by at least one fastening element so as to have the pressure-bearing portion tightly bear against the chair back structure; and
a pressing member, having an upwardly protruding rounded blunt shape, disposed at a top portion of the fastener.

2. The abdominal thrust device of claim 1, wherein the at least one fastening element is at least one selected from a group consisting of a screw, an adhesive, a stopper portion, a spring, an extending clamping portion, and a protruding portion.

3. The abdominal thrust device of claim 2, wherein the fastening element is a screw, and an end of the screw is provided with a stopper plate, the screw bearing against the chair back structure via the stopper plate.

4. The abdominal thrust device of claim 1, wherein the fastener comprises a fixing plate, the pressing member is disposed at a top portion of the fixing plate, and a bottom of the fixing plate serves as the pressure-bearing portion.

5. The abdominal thrust device of claim 1, wherein the pressing member has a shape of a human fist.

6. The abdominal thrust device of claim 1, wherein a maximum width of a horizontal cross-section of the pressing member is, for example, greater than or equal to 8 cm, preferably greater than or equal to 10 cm, and more preferably greater than or equal to 12 cm, the horizontal cross-section being parallel to an upper edge where the pressure-bearing portion tightly bears the chair back structure.

7. The abdominal thrust device of claim 1, further comprises a soft material that covers at least a portion of the pressing member.

8. The abdominal thrust device of claim 1, wherein the top portion of the fastener has a first engaging portion, and a bottom of the pressing member has a second engaging portion, the first engaging portion engaging with the second engaging portion to fix the pressing member to the top portion of the fastener.

9. The abdominal thrust device of claim 1, wherein the fastener, the pressing member, or the at least one fastening element are further connected to each other by a chain or a rope.

10. The abdominal thrust device of claim 1, wherein the fastener further comprises a first side extension wall, the first side extension wall being connected to a side edge of the pressure-bearing portion and disposed perpendicular to the pressure-bearing portion;
the first side extension wall and a remaining portion of the fastener other than the first side extension wall are either integrally formed or each is a separate component connected to each other.

11. The abdominal thrust device of claim 10, wherein the fastener further comprises a second side extension wall, the second side extension wall being connected to another side edge of the pressure-bearing portion opposite to the side edge and disposed perpendicular to the pressure-bearing portion;
the second side extension wall and a remaining portion of the fastener other than the second side extension wall are either integrally formed or each is a separate component connected to each other.

12. The abdominal thrust device of claim 11, wherein the at least one fastening element is at least one selected from a group consisting of a screw, an adhesive, a stopper portion, and a spring.

13. The abdominal thrust device of claim 10, wherein the chair back structure is a chair back crest rail;
the fastener further comprises a horizontal extension wall, the horizontal extension wall being connected to the first side extension wall and disposed parallel to the pressure-bearing portion.

14. The abdominal thrust device of claim 13, wherein the at least one fastening element is an extending clamping portion or a spring, the extending clamping portion or the spring connecting the first side extension wall and the horizontal extension wall.

15. The abdominal thrust device of claim 1, wherein the fastener is a C-shaped clamp.

16. The abdominal thrust device of claim 15, wherein the at least one fastening element is a screw.

17. The abdominal thrust device of claim 15, wherein the C-shaped clamp elastically clamps the chair back structure.

18. The abdominal thrust device of claim 17, wherein the at least one fastening element is a protruding portion, and the C-shaped clamp bears against the chair back structure by the protruding portion.

19. The abdominal thrust device of claim 18, wherein the at least one fastening element and the fastener are integrally formed.

20. The abdominal thrust device of claim 15, wherein the C-shaped clamp comprises at least one side extension wall.
